(19) United States
(12) Patent Application Publication
(10) Pub. No.: US 2023/0413464 A1
(43) Pub. Date: Dec. 21, 2023

(54) CIRCUIT HOUSING FOR WEARABLE INTRAORAL APPLICATION

(71) Applicant: Ivoclar Vivadent AG, Schaan (LI)

(72) Inventors: Alexander Skawran, Sargans (CH); Marvin Kuhn, Feldkirch (AT); Kai Rist, Feldkirch (AT)

(21) Appl. No.: 18/325,818

(22) Filed: May 30, 2023

(30) Foreign Application Priority Data
Jun. 21, 2022 (EP) 22180128.5

(51) Int. Cl. H05K 5/06; A61B 5/00

(52) U.S. Cl. CPC H05K 5/06; A61B 5/682; A61B 5/0002; A61B 2562/16

(57) ABSTRACT

A circuit housing (100) for a wearable intraoral application, including an inner layer (105-1) of a first material (101-1) for waterproof enclosure of an electronic circuit (103); and an outer layer (105-2) of a second material (101-2) for spatially fitting the circuit housing to a set of teeth (111), at least partially covering the first material (101-1).

### CROSS-REFERENCE TO RELATED APPLICATIONS

This application claims priority to European Patent Application No. 22180128.5 filed on Jun. 21, 2022, the disclosure of which is incorporated herein by reference in its entirety.

### TECHNICAL FIELD

The present invention relates to a circuit housing for a wearable intraoral application and a method of manufacturing a circuit housing for a wearable intraoral application.

### BACKGROUND

U.S. Pat. No. 4,629,424 A, which is hereby incorporated by reference, relates to a device for intraoral environmental sensing comprising a removable oral device with a number of chemically sensitive electrodes and a common reference electrode at the chemical sensing sites.

Currently, electronic devices for an intraoral space are protected by an outer, separate housing made of polymers. New materials, on the other hand, allow smaller sizes of electronic devices for the dental area. These housings often leak and provide inadequate protection for the internal electronic system.

U.S. Ser. Nos. 11/638,535, 11/504,060, 10/729,372, 10/314,537, and 10/178,946 are directed to intraoral sensors and are hereby incorporated by reference in their entirety.

### SUMMARY

It is the technical object of the present invention to provide a circuit housing that can better protect an electronic circuit in an intraoral space from moisture.

This technical object is solved by subject matter according to the independent claims. Technically advantageous embodiments are the subject-matter of the dependent claims, the description and the drawings.

According to a first aspect, the technical object is solved by a circuit housing for a wearable intraoral application, comprising an inner layer of a first material for waterproof enclosure of an electronic circuit; and an outer layer of a second material for spatially fitting the circuit housing to a set of teeth, at least partially covering the first material.

The circuit housing achieves the technical advantage that water ingress into the circuit housing due to a chewing movement can be prevented. In addition, the second layer serves as mechanical protection against damage to the first layer. The circuit housing with two separate layers is more robust and less susceptible to water ingress. The circuit housing can protect electronic circuits for medical and dental applications from external influences such as water, saliva, other liquids or contamination and prevent failure.

In a technically advantageous embodiment of the circuit housing, the first material of the inner layer comprises at least one protective varnish consisting of silicone, (meth)acrylate resin, urethane resin, epoxy resin, parylene or mixtures thereof. This provides, for example, the technical advantage of using particularly suitable materials for waterproof enclosure of the electronic circuit.

In a further technically advantageous embodiment of the circuit housing, the second material of the outer layer comprises at least one 3D-printable material (for example in stereolithography), in particular light-polymerizable resins, preferably (meth)acrylates, epoxy resins, urethane resins, thermoplastics, and/or the second material comprises a millable material, in particular poly(meth)acrylate, polycarbonate or ceramic. The second material may be formed from a material that can be used in a three-dimensional printing process or milling process. This provides the technical advantage, for example, that the circuit housing can be easily adapted spatially to different intraoral spaces.

In preferred embodiments, materials for the second material of the outer layer are orthodontic splint (e.g. bite splint) materials such as ProArt Print Splint from Ivoclar Vivadent.

In another technically advantageous embodiment of the circuit housing, the second material has a fracture toughness of greater than 0.5 MPa\sqrt{m} or a flexural modulus of less than 2500 MPa. This provides the technical advantage, for example, that the second material adapts well to a set of teeth.

In another technically advantageous embodiment of the circuit housing, the first and second materials permit transmission of electromagnetic radiation in the range between 2.2 and 2.4 GHz. This provides the technical advantage, for example, that data can be transmitted through the circuit housing via Bluetooth.

In another technically advantageous embodiment of the circuit housing, the first and second materials permit transmission of electromagnetic radiation in the range between 13 and 14 MHz. This provides the technical advantage, for example, that data can be transmitted through the circuit housing via near field communication (NFC).

In another technically advantageous embodiment of the circuit housing, the first and second materials have an electrical conductivity below 10⁻¹⁰ S/m. This provides the technical advantage, for example, that leakage currents can be prevented.

In another technically advantageous embodiment of the circuit housing, the circuit housing comprises an opening for a sensor. This provides the technical advantage, for example, that saliva can be introduced to the sensor for measurement.

In another technically advantageous embodiment of the circuit housing, the circuit housing comprises an electronic circuit with a device with a number of chemical, biological and/or physical sensors. Examples include a circuit having one or more of a pH sensor, a lactate sensor, a temperature sensor, a glucose sensor, a volatile sulfur compound sensor, an alcohol sensor, an atmospheric pressure sensor, a cortisol sensor, an osmolality sensor, an ion-selective sensor, an acceleration sensor, a pressure sensor, and/or a moisture sensor. This provides the technical advantage, for example, of using particularly suitable sensors for an intraoral space. The circuit housing may also comprise multiple sensors. For example, in the case of a lactate or pH value measurement, two identical sensors can also be used in a circuit in order to generate measured values at different points or as a check of the first sensor.

In a technically advantageous embodiment of a circuit housing assembly, the circuit housing assembly comprises the circuit housing and an electronic circuit having a device with a number of chemical, biological, and/or physical sensors. Examples include a circuit having one or more of a pH sensor, a lactate sensor, a temperature sensor, a glucose sensor, a volatile sulfur compound sensor, an alcohol sensor, an atmospheric pressure sensor, a cortisol sensor, an osmolality sensor, an ion-selective sensor, an acceleration sensor, a pressure sensor, and/or a moisture sensor. This provides the technical advantage, for example, of using particularly suitable sensors for an intraoral space. The circuit housing assembly may also comprise multiple sensors. For example, in the case of a lactate or pH value measurement, two identical sensors can also be used in a circuit in order to generate measured values at different points or as a check of the first sensor.

In another technically advantageous embodiment of the circuit housing, the circuit housing comprises an electronic circuit with a circuit section for wireless transmission of energy, a transceiver unit for wireless data transmission and/or an energy storage. This provides the technical advantage, for example, that energy and/or data can be transmitted to the electronic circuit.

In another technically advantageous embodiment of a circuit housing assembly, the circuit housing assembly comprises the circuit housing and an electronic circuit with a circuit section for wireless transmission of energy, a transceiver unit for wireless data transmission and/or an energy storage. This provides the technical advantage, for example, that energy and/or data can be transmitted to the electronic circuit.

In another technically advantageous embodiment of the circuit housing, the circuit housing comprises an intermediate layer for connecting the inner layer and the outer layer. This provides the technical advantage, for example, of improving adhesion between the inner and outer layers or increasing mechanical protection of the inner components.

In another technically advantageous embodiment of the circuit housing, the circuit housing comprises an electronic circuit molded into the first material. This provides the technical advantage, for example, of further improving moisture protection.

In another technically advantageous embodiment of a circuit housing assembly, the circuit housing assembly comprises the circuit housing and an electronic circuit molded into the first material. This provides the technical advantage, for example, of further improving moisture protection.

According to a second aspect, the technical object is solved by a method of manufacturing a circuit housing for a wearable intraoral application, comprising the steps of arranging an inner layer of a first material for waterproof enclosure of an electronic circuit; and arranging an outer layer of a second material for spatially fitting the circuit housing to a set of teeth, at least partially covering the first material. Thereby, the same technical advantages are achieved as by the circuit housing according to the first aspect.

In a technically advantageous embodiment of the method, the electronic circuit is molded into the first material. This provides the technical advantage, for example, that moisture penetration is particularly effectively prevented.

In another technically advantageous embodiment of the method, the first material is varnished onto the electronic circuit or applied by physical vapor deposition or chemical vapor deposition. This provides the technical advantage, for example, that the electronic circuit can be enclosed with a thin film in a particularly water-resistant manner.

### BRIEF DESCRIPTION OF THE DRAWINGS

Exemplary embodiments of the invention are shown in the drawings and are described in more detail below, in which:

FIG. 1 shows a schematic illustration of a circuit housing in a first embodiment;

FIG. 2 shows a schematic illustration of a circuit housing in a second embodiment; and

FIG. 3 shows a block diagram of a method for manufacturing a circuit housing.

### DETAILED DESCRIPTION

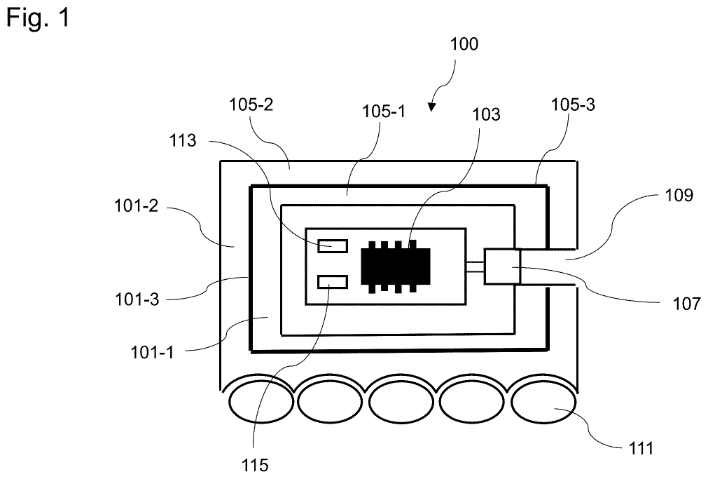

FIG. 1 shows a schematic illustration of a circuit housing 100 for a wearable intraoral electronic circuit 103. The circuit housing 100 can be used in a patient's mouth as a wearable device.

The electronic circuit 103 used within the circuit housing 100 is worn intraorally for a period of several minutes to days. The circuit housing 100 protects the electronic circuit 103 from saliva and ingested food or liquid. In addition, the circuit housing 100 protects the electronic circuit 103 from being damaged by accidental biting. The circuit housing 100 thus provides a seal for the electronic circuit 103 from external influences. Materials that are already approved for use in intraoral applications may be used for the sealing. Since the circuit housing 100 is worn in a mouth, biocompatible materials may be used for this purpose. The EN ISO 7405:2019 standard is used to evaluate the biocompatibility of dental materials.

The protection of the electronic circuit 103 follows a layering principle. The circuit housing 100 includes an inner layer 105-1 made of a biocompatible material 101-1 for waterproof enclosure of an electronic circuit 103.

On top of the inner layer is an outer layer 105-2 of a biocompatible material 101-2 for spatially fitting the circuit housing to a set of teeth 111. The outer layer at least partially covers the first material 101-1 of the inner layer 105-1 and thereby surrounds the first material 101-1.

The outer layer 105-2 may be custom-made to fit the patient and seamlessly fit to the set of teeth 111 so that it resists external pressure when teeth are clenched. Shaping additive or subtractive manufacturing processes are therefore advantageous for manufacturing the outer layer 105-2, as they can produce the outer layer 105-1 in the desired spatial shape. Three-dimensional printing or injection molding processes are suitable for this purpose.

The material 101-1 of the inner layer 105-1 is formed, for example, by a protective varnish (conformal coating). The protective varnish may consist of silicone, (meth)acrylate resin, urethane resin, epoxy resin, parylene, or mixtures thereof. The material 101-1 of the inner layer 105-1 may also comprise polyvinylsiloxane. In preferred embodiments, materials for the material 101-1 of the inner layer 105-1 are polyvinylsiloxane impression materials such as those of the Virtual™ product line of the Ivoclar Vivadent company Putty, Heavy Body, Monophase, Light Body and Extra Light Body, or (meth)acrylate resins such as Heliobond™ resin of the Ivoclar Vivadent company.

The inner layer 105-1 should be as thin as possible. For example, the inner layer 105-1 has a minimum thickness of preferably 5 mm to 0.0001 mm, very preferably 3 mm to 0.001, particularly preferably 1 mm to 0.01 mm. For example, the inner layer 105-1 has a maximum thickness of preferably 10 mm to 0.0001 mm, very preferably 6 mm to 0.001, particularly preferably 2 mm to 0.01 mm. For example, the outer layer 105-2 has a minimum thickness of preferably 15 mm to 0.1 mm, very preferably 12 mm to 0.5 mm, particularly preferably 10 mm to 1 mm. For example, the outer layer 105-2 has a maximum thickness of preferably 30 mm to 0.1 mm, very preferably 20 mm to 0.5 mm, particularly preferably 15 mm to 1 mm.

The material 101-2 of the outer layer 105-2 comprises a material that can be used in a three-dimensional printing process, such as a radical polymerizable composition or a light polymerizable resin for stereolithography. The material 101-2 of the outer layer 105-2 may comprise at least one 3D printable material, in particular light polymerizable resins, preferably (meth)acrylates, epoxy resins, urethane resins, and/or thermoplastics. The second material 101-2 may also comprise a millable material, in particular poly(meth)acrylate, polycarbonate, or ceramic.

The circuit housing 100 may further comprise an intermediate layer 101-3 as a bonding material between the inner layer 101-1 and the outer layer 101-2. The material 105-3 of the intermediate layer 101-3 serves as a bonding material and is a potting material or potting compound, such as silicone, urethane resin, epoxy resin, or (meth)acrylate resin.

The electronic circuit 103 comprises a transceiver unit 113 for wireless data transmission, such as a communication unit for wireless data transmission such as Bluetooth or NFC. To enable this data transmission, the materials 101-1, 101-2, and 101-3 of the circuit housing 100 are transparent to electromagnetic radiation in the range between 2.2 and 2.4 GHz (Bluetooth) or in the range between 13 and 14 MHz (NFC). In addition, the electronic circuit 103 may comprise a wirelessly rechargeable battery as an energy storage 115. To this end, the electronic circuit 103 has a circuit section for wirelessly transmitting energy, for example, using an induction loop. Therefore, the materials 101-1, 101-2, and 101-3 used allow wireless charging and communication via Bluetooth or NFC.

The circuit housing 100 may comprise electrical outputs or leads for various sensors 107 of the electronic circuit 103. For example, the sensors 107 are for determining the amount of various substances, such as lactate, glucose, cortisol, alcohol, or volatile sulfur compounds (VSCs). The sensors 107 may further be provided for measuring a pH, a temperature, an atmospheric pressure, or an osmolality.

To be able to measure these substances and values, the sensors 107 are in direct contact with the saliva. Depending on the application, the sensors 107 are not encased to allow saliva or air to come into contact with the sensor 107. To this end, the circuit housing 100 comprises at least one opening 109 extending through the layers 101-1, 101-2, and 101-3 to allow the fluid to reach the sensor 107. The openings 109 of the different sensors 107 may differ in shape, size and position and may be adapted to the individual circuit housing 100 of the wearer.

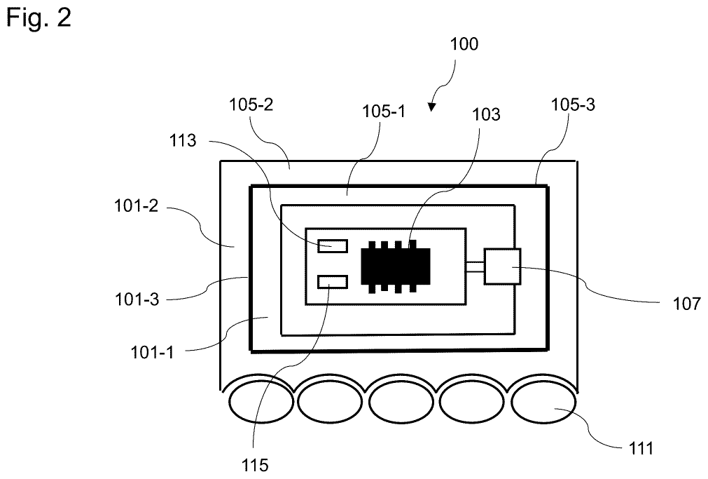

FIG. 2 shows a schematic illustration of a circuit housing 100 in a second embodiment. In this embodiment, the inner layer 105-1, the intermediate layer 105-3 and the outer layer 105-2 are completely closed around the electronic circuit 103.

Accordingly, the circuit housing 100 does not have an opening 109.

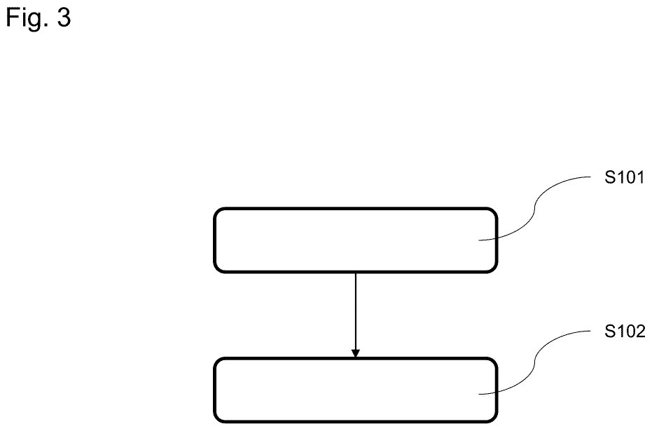

FIG. 3 shows a block diagram of a method for manufacturing a circuit housing 100 for a wearable intraoral application. In a first step S101, an inner layer of the first material 101-1 is arranged for waterproof enclosure of the electronic circuit 103. For example, the electronic circuit 103 is molded in silicone or another material to keep it waterproof. This encasement forms the first protective layer for protecting the electronic circuit 103.

In a further step S102, the outer layer of the second material 101-2 is arranged to spatially fit the circuit housing to the set of teeth 111, which at least partially covers the first material 101-1.

The inner layer can be varnished on, for example. Preferably, the inner layer 105-1 is not printed, but is applied to the electronic circuit 103 by encasing/encapsulating such as casting, potting, dip coating, spray coating, brush coating, physical vapor deposition (PVD), or chemical vapor deposition (CVD). Generally, the inner layer 105-1 can be cured thermally, chemically, or by UV radiation or air drying. These processes can be handled in a simple manner, and the electronic circuit 103 can be protected with a thin film as the inner layer 101-1. The electronic circuit 103 can be sealed only partially or completely, as required.

Depending on the size of the electronic circuit 103, it can also be immersed in the material 101-1 or painted or coated with it. Once the material 101-1 has cured, the electronic circuit 103 is surrounded by a protective layer. Multiple layers of the material 101-1 may also be applied for sealing. After a complete sealing, the electronic circuit 103 is enclosed in a waterproof manner.

After the first step S101, a flexible and bonding intermediate layer 101-3, such as an adhesive, may also be initially applied to bond the inner layer 101-1 to the outer layer 101-2. The intermediate layer 101-3 also forms further mechanical protection for the underlying layer 101-1 and the electronic circuit. The intermediate layer 101-3 may be formed of a third material as a bonding material, such as polyvinyl ether or silicone, in particular addition silicone or vinyl polysiloxane.

In a dental application, the sealed electronic circuit 103 can withstand saliva and other fluids in the mouth without damage. The materials 101-1, 101-2, and 101-3 can be removed from the electronic circuit 103 as needed.

All of the features explained and shown in connection with individual embodiments of the invention may be provided in different combinations in the subject matter of the invention to simultaneously realize their beneficial effects.

All method steps can be implemented by devices which are suitable for executing the respective method step. All functions that are executed by the features of the subject matter can be a method step of a method.

The scope of protection of the present invention is given by the claims and is not limited by the features explained in the description or shown in the figures.

### REFERENCE SIGN LIST

100 circuit housing
101 material
103 electronic circuit
105 layer
107 sensor
109 opening
111 set of teeth
113 transceiver unit
115 energy storage

1. A circuit housing for a wearable intraoral application, comprising
an inner layer of a first material for waterproof enclosure of an electronic circuit; and
an outer layer of a second material for spatially fitting the circuit housing to a set of teeth, which at least partially covers the first material.

2. The circuit housing according to claim 1,
wherein the first material comprises at least one protective varnish comprising silicone, (meth)acrylate resin, urethane resin, epoxy resin, parylene, or mixtures thereof.

3. The circuit housing according to claim 1,
wherein the second material comprises at least one 3D-printable material comprising light-polymerizable resin comprising (meth)acrylate resin, epoxy resin, urethane resin, thermoplastic or a mixture thereof

4. The circuit housing according to claim 1,
wherein the second material comprises a millable material comprising poly(meth)acrylate, polycarbonate or ceramic.

5. The circuit housing according to claim 1,
wherein the second material has a fracture toughness of greater than 0.5 MPa\sqrt{m} or a flexural modulus of less than 2500 MPa.

6. The circuit housing according to claim 1,
wherein the first and second materials allow transmission of electromagnetic radiation in the range between 2.2 and 2.4 GHz.

7. The circuit housing according to claim 1,
wherein the first and second materials allow transmission of electromagnetic radiation in the range between 13 and 14 MHz.

8. The circuit housing according to claim 1,
wherein the first and second materials have an electrical conductivity below 10⁻¹⁰ S/m.

9. The circuit housing according to claim 1,
wherein the circuit housing comprises an opening for a sensor.

10. The circuit housing according to claim 1,
wherein the circuit housing comprises an electronic circuit having a pH sensor, a lactate sensor, a temperature sensor, a glucose sensor, a volatile sulfur compound sensor, an alcohol sensor, an atmospheric pressure sensor, a cortisol sensor, an osmolality sensor, an ion-selective sensor, an acceleration sensor, a pressure sensor, and/or a moisture sensor.

11. The circuit housing according to claim 1,
wherein the circuit housing comprises an electronic circuit for wireless transmission of energy, a transceiver unit for wireless data transmission and/or an energy storage.

12. The circuit housing according to claim 1,
wherein the circuit housing comprises an intermediate layer for connecting the inner layer and the outer layer.

13. The circuit housing according to claim 1,
wherein the circuit housing comprises an electronic circuit molded into the first material.

14. A method of manufacturing a circuit housing for a wearable intraoral application, comprising the steps of:
arranging an inner layer of a first material for waterproof enclosure of an electronic circuit; and
arranging an outer layer of a second material for spatially fitting the circuit housing to a set of teeth, which at least partially covers the first material.

15. The method according to claim 14,
wherein the electronic circuit is molded into the first material.

16. The method according to claim 14,
wherein the first material is varnished onto the electronic circuit or applied by physical vapor deposition (PVD) or chemical vapor deposition (CVD).
